(19) United States
(12) Patent Application Publication
(10) Pub. No.: US 2024/0148446 A1
(43) Pub. Date: May 9, 2024

(54) CONTROL SYSTEM AND METHOD FOR NAVIGATION AND REDUCTION OPERATION

(71) Applicant: BEIJING ROSSUM ROBOT TECHNOLOGY CO., LTD, Haidian District, Beijing (CN)

(72) Inventors: Xinbao WU, Haidian District, Beijing (CN); Chunpeng ZHAO, Haidian District, Beijing (CN); Shuchang SHI, Haidian District, Beijing (CN); Lijun SHI, Haidian District, Beijing (CN); Ke XU, Haidian District, Beijing (CN); Xiangrui ZHAO, Haidian District, Beijing (CN)

(21) Appl. No.: 18/548,354

(22) PCT Filed: May 5, 2022
(86) PCT No.: PCT/CN2022/090974

§ 371 Date: Aug. 30, 2023

(30) Foreign Application Priority Data
Jul. 9, 2021 (CN) 202110779036.4

(51) Int. Cl. A61B 34/20; A61B 34/10; A61B 90/00; G06T 7/60; G06T 7/70

(52) U.S. Cl. CPC A61B 34/20; A61B 34/10; A61B 90/37; G06T 7/60; G06T 7/70; A61B 2034/107; A61B 2034/2055; A61B 2090/3762

(57) ABSTRACT

Control system for navigation and reduction operation including a master control apparatus having a host and an optical tracker; a tracing apparatus including a target body tracer arranged on a target body; the optical tracker is configured to obtain a geometric feature of the target body tracer in an actual working space; the host is configured to convert a preliminary image into an intermediate image by matching the preliminary image with the intermediate image, and to convert the preliminary image into the actual working space according to a geometric feature of the target body tracer in the intermediate image and the geometric feature of the target body tracer in the actual working space, to obtain a target pose of the operation apparatus in the actual working space, and to control an operation apparatus to move to the target pose for reduction. A method for navigation and reduction operation is provided.

### TECHNICAL FIELD

The present disclosure relates to the technical field of navigation and reduction robots, in particular to a control system and method for navigation and reduction operation.

### BACKGROUND

At present, a closest navigation solution is to directly use an intraoperative image and convert it into a real surgical space, which is easily affected by a range of the intraoperative image. The navigation solution is unable to provide real-time tracking of a bone fragment in a fracture and cannot be directly used for reduction of the fracture.

An existing solution for reduction of a fracture mainly includes the determination of a fracture reduction condition according to intraoperative X-ray through continuous adjustment and a large amount of radioactivity, and is unable to implement precise reduction as a three-dimensional pose cannot be fully expressed with two-dimensional information. Another solution is to cut open a fractured site and implement reduction in a visible condition, which leads to a large wound and does not contribute to the recovery of a patient. Therefore, in the prior art, for a hidden and difficultly exposed target body whose position state cannot be intuitively obtained, there are no control system capable of implementing real-time tracking and no control system capable of operating the target body to be precisely reduced, whether in the actual industrial or medical field.

### SUMMARY

The present disclosure provides a control system and method for navigation and reduction operation, which are used to overcome the defect of inability to track a hidden and difficultly exposed target body whose position state cannot be intuitively obtained in real time in the prior art, implement real-time three-dimensional navigation, and can track the target body in real time and feed it back to an operator in the form of a three-dimensional model and a preliminary image, so as to precisely reduce and position the target body during actual work.

The present disclosure provides a control system for navigation and reduction operation, including a master control apparatus, a tracing apparatus, and an operation apparatus, where the master control apparatus includes a host and an optical tracker; the tracing apparatus includes a target body tracer arranged on a target body; the optical tracker is configured to obtain a geometric feature of the target body tracer in an actual working space; the host is configured to convert a preliminary image into an intermediate image by matching the preliminary image with the intermediate image, and to convert the preliminary image into the actual working space according to a geometric feature of the target body tracer in the intermediate image and the geometric feature of the target body tracer in the actual working space; and the host is further configured to obtain a target pose of the operation apparatus in the actual working space, and to control the operation apparatus to move to the target pose for reduction.

According to the control system for navigation and reduction operation provided by the present disclosure, the optical tracker is further configured to obtain a pose of the target body tracer in the actual working space in real time; and the host is further configured to convert the preliminary image into the actual working space in real time, and to obtain a pose of the target body in the actual working space.

According to the control system for navigation and reduction operation provided by the present disclosure, the tracing apparatus further includes a tool tracer arranged on a tool; the optical tracker is further configured to obtain a pose of the tool tracer in the actual working space in real time; and the host is further configured to obtain a pose of the tool in the actual working space.

According to the control system for navigation and reduction operation provided by the present disclosure, the tracing apparatus further includes an operation apparatus tracer arranged on the operation apparatus; the optical tracker is further configured to obtain a pose of the operation apparatus tracer in the actual working space in real time; and the host is further configured to obtain a pose of the operation apparatus in the actual working space, to obtain the target pose of the operation apparatus in the actual working space according to a target pose of the target body in the actual working space, and to control the operation apparatus to move to the target pose for reduction.

According to the control system for navigation and reduction operation provided by the present disclosure, the operation apparatus includes a robotic arm controller and a robotic arm having six or more degrees of freedom; the robotic arm is connected to the robotic arm controller; the robotic arm controller is connected to the host; and the operation apparatus tracer is arranged on the robotic arm.

The present disclosure further provides a method for navigation and reduction operation performed using the control system for navigation and reduction operation as described above, the method including:
obtaining a preliminary image and an intermediate image;
converting the preliminary image into the intermediate image according to the preliminary image and the intermediate image;
obtaining a geometric feature of a target body tracer in an actual working space;
converting the preliminary image into the actual working space according to a geometric feature of the target body tracer in the preliminary image and the geometric feature of the target body tracer in the actual working space; and
obtaining a target pose of an operation apparatus in the actual working space, and controlling the operation apparatus to move to the target pose for reduction.

According to the method for navigation and reduction operation provided by the present disclosure, the method further includes:
obtaining a real-time pose of the target body tracer; and
obtaining a three-dimensional model for a target body on the basis of converting the preliminary image into the actual working space.

According to the method for navigation and reduction operation provided by the present disclosure, the method further includes:
obtaining a real-time pose of a tool tracer; and
obtaining a pose of the tool in the actual working space and a three-dimensional model for the tool according to a relative pose of the tool tracer and the tool and the real-time pose of the tool tracer.

According to the method for navigation and reduction operation provided by the present disclosure, the method further includes:
obtaining a real-time pose of an operation apparatus tracer; and
obtaining a pose of the operation apparatus in the actual working space according to a relative pose of the operation apparatus tracer and the operation apparatus and the real-time pose of the operation apparatus tracer.

According to the method for navigation and reduction operation provided by the present disclosure, the method further includes:
obtaining a target pose of the target body in the actual working space;
obtaining the target pose of the operation apparatus according to a connection relationship between the target body and the operation apparatus; and
controlling the operation apparatus to move to the target pose.

According to the control system for navigation and reduction operation provided by the present disclosure, the target body tracer is fixed to the target body that needs to be tracked; the host obtains the intermediate image and the preliminary image, and converts the preliminary image into a space of the intermediate image by matching the intermediate image with the preliminary image; the optical tracker obtains the geometric feature of the target body tracer in the actual working space and sends it to the host; and the host obtains a conversion relationship between the intermediate image and the actual working space according to the geometric feature of the target body tracer in the intermediate image and the geometric feature of the target body tracer in the actual working space that is obtained by the optical tracker, and further converts the preliminary image into the actual working space. The host of the master control apparatus converts the preliminary image into the actual working space by matching the intermediate image with the preliminary image, and the optical tracker is used to track it in real time, so that the real-time three-dimensional navigation is implemented, and the target body can be tracked in real time and is fed back to the operator in the form of the three-dimensional model and the preliminary image; the host obtains the pose of the operation apparatus in the actual working space, and calculates the target pose of the operation apparatus according to a reduction plan, the real-time pose of the target body tracer, and the current connection relationship between the target body and the operation apparatus; through communication with the operation apparatus, a command is sent to control the operation apparatus to move to the target pose and monitor a motion state of the operation apparatus in real time; and the target body is operated by the operation apparatus to be precisely reduced, so as to precisely reduce and position the target body during actual work.

In addition to the technical problems solved by the present disclosure, the technical features of the formed technical solutions, and the advantages brought by the technical features of these technical solutions described above, other technical features of the present disclosure and the advantages brought by these technical features will be further described in conjunction with the accompanying drawings or learned through the practice of the present disclosure.

### BRIEF DESCRIPTION OF THE DRAWINGS

To more clearly illustrate the technical solutions in the embodiments of the present disclosure or in the prior art, the accompanying drawings that need to be used in the description of the embodiments or the prior art will be briefly described below. Apparently, the accompanying drawings in the description below merely illustrate some embodiments of the present disclosure. Those of ordinary skill in the art may also derive other accompanying drawings from these accompanying drawings without creative efforts.

FIG. 1 is a schematic structural diagram of a control system for navigation and reduction operation provided by the present disclosure.

### IN REFERENCE SIGNS

100: master control apparatus; 110: host; 120: optical tracker; 130: display; 140: keyboard and mouse unit; 200: tracing apparatus; 210: target body tracer; 220: tool tracer; 230: operation apparatus tracer; 300: preoperative imaging device; 400: intraoperative imaging device; 500: tool; 600: operation apparatus; 610: robotic arm; 620: robotic arm controller; and 700: target body.

### Detailed Description of the Embodiments

The implementation of the present disclosure will be further described in detail in combination with the accompanying drawings and the embodiments. The embodiments below are used to illustrate the present disclosure, but not intended to limit the scope of the present disclosure.

In the description of the embodiments of the present disclosure, it should be noted that the orientations or positional relationships indicated by the terms “center”, “longitudinal”, “transverse”, “upper”, “lower”, “front”, “back”, “left”, “right”, “vertical”, “horizontal”, “top”, “bottom”, “inside”, “outside”, etc. are based on the orientations or positional relationships shown in the accompanying drawings, are only for the convenience of describing the embodiments of the present disclosure and simplifying the description rather than indicating or implying that the apparatus or element referred to must have a specific orientation or be constructed and operated in a specific orientation, and therefore should not be construed as a limitation to the embodiments of the present disclosure. Furthermore, the terms “first”, “second”, and “third” are merely used for descriptive purposes and should not be understood to indicate or imply relative importance.

In the description of the embodiments of the present disclosure, it should be noted that the terms “connected” and “connection” should be understood in a broad sense, unless otherwise expressly specified and defined. For example, the “connection” may be a fixed connection, a detachable connection, or an integrated connection; the “connection” may also be a mechanical connection or an electrical connection; and the “connected” may be directly connected or indirectly connected via an intermediate medium. Those of ordinary skill in the art may understand the specific meanings of the above terms in the embodiments of the present disclosure according to the specific circumstances.

In the embodiments of the present disclosure, the state that the first feature is “over” or “under” the second feature may include a state that the first and second features are in direct contact with each other, or a state that the first and second features are in indirect contact with each other via an intermediate medium, unless otherwise expressly specified and defined. Moreover, the state that the first feature is “over”, “above”, and “on” the second feature may include a state that the first feature is right above or obliquely above the second feature, or only indicates that the horizontal height of the first feature is greater than the horizontal height of the second feature. The state that the first feature is “under”, “below”, and “beneath” the second feature may include a state that the first feature is right below or obliquely below the second feature, or only indicates that the horizontal height of the first feature is smaller than the horizontal height of the second feature.

In the description of this specification, the description with reference to the term such as “one embodiment”, “some embodiments”, “an example”, “a specific example”, or “some examples” means that the specific features, structures, materials, or characteristics described in combination with the embodiments or examples are included in at least one embodiment or example of the embodiments of the present disclosure. In this specification, the schematic representations of the above terms need not be directed to the same embodiments or examples. Furthermore, the particular features, structures, materials, or characteristics described may be combined in a suitable manner in any one or more embodiments or examples. In addition, different embodiments or examples described in this specification and features of different embodiments or examples may be connected and combined by those skilled in the art without mutual contradiction.

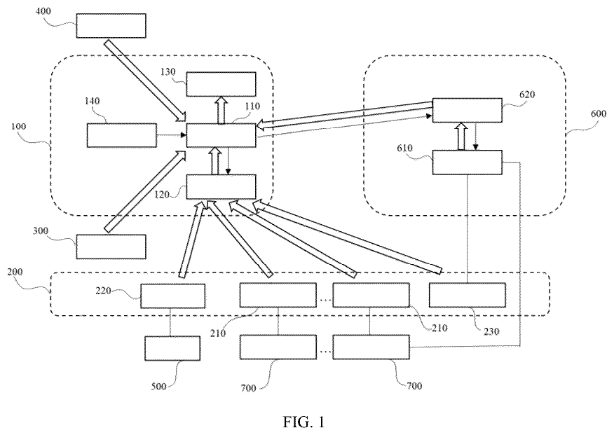

As shown in FIG. 1, a control system for navigation and reduction operation provided by an embodiment of the present disclosure includes a master control apparatus 100, a tracing apparatus 200, and an operation apparatus 600, where the master control apparatus 100 includes a host 110 and an optical tracker 120; the tracing apparatus 200 includes a target body tracer 210 arranged on a target body 700; the optical tracker 120 is configured to obtain a geometric feature of the target body tracer 210 in an actual working space; the host 110 is configured to convert a preliminary image into an intermediate image by matching the preliminary image with the intermediate image, and to convert the preliminary image into the actual working space according to a geometric feature of the target body tracer 210 in the intermediate image and the geometric feature of the target body tracer 210 in the actual working space; and the host 110 is further configured to obtain a target pose of the operation apparatus 600 in the actual working space, and to control the operation apparatus 600 to move to the target pose for reduction.

According to the control system for navigation and reduction operation provided by this embodiment of the present disclosure, the target body tracer 210 is fixed to the target body 700 that needs to be tracked; the host 110 obtains the intermediate image and the preliminary image, and converts the preliminary image into a space of the intermediate image by matching the intermediate image with the preliminary image; the optical tracker 120 obtains the geometric feature of the target body tracer 210 in the actual working space and sends it to the host 110; and the host 110 obtains a conversion relationship between the intermediate image and the actual working space according to the geometric feature of the target body tracer 210 in the intermediate image and the geometric feature of the target body tracer 210 in the actual working space that is obtained by the optical tracker 120, and further converts the preliminary image into the actual working space. The host 110 of the master control apparatus 100 converts the preliminary image into the actual working space by matching the intermediate image with the preliminary image, and the optical tracker 120 is used to track it in real time, so that real-time three-dimensional navigation is implemented, and the target body 700 can be tracked in real time and is fed back to an operator in the form of a three-dimensional model and the preliminary image; the host 110 obtains a pose of the operation apparatus 600 in the actual working space, and calculates the target pose of the operation apparatus 600 according to a reduction plan, a real-time pose of the target body tracer 210, and a current connection relationship between the target body 700 and the operation apparatus 600; through communication with the operation apparatus 600, a command is sent to control the operation apparatus 600 to move to the target pose and monitor a motion state of the operation apparatus 600 in real time; and the target body 700 is operated by the operation apparatus 600 to be precisely reduced, so as to precisely reduce and position the target body 700 during actual work.

When this embodiment is applied to the reduction of a bone fragment of a patient with a fracture through the medical technology, the master control apparatus 100 may be a master control trolley, and the host 110 may be controlled by a keyboard and mouse unit 140. A preoperative imaging device 300, such as a CT machine, acquires a preoperative image of a fractured site of the patient on admission as the preliminary image and sends it to the host 110, where the preliminary image may be a three-dimensional image. An intraoperative imaging device 400, such as the CT machine, acquires an intraoperative image of the fractured site of the patient during operation as the intermediate image and sends it to the host 110, where the intermediate image may be a two-dimensional or three-dimensional image. The actual working space is a surgical space. FIG. 1 shows a schematic structural diagram of a control system for navigation and reduction operation, where a straight line for connection represents a fixed connection, a solid arrow represents a control flow, and a hollow arrow represents a data flow.

Firstly, the target body tracer 210 is fixed to the bone fragment that needs to be tracked, the preoperative imaging device 300 obtains the preoperative image and transmits it to the host 110, the intraoperative imaging device 400 obtains the intraoperative image and transmits it to the host 110, and the host 110 converts the preoperative image into the space of the intraoperative image by matching the preoperative image with the intraoperative image through image or point cloud registration. The host 110 obtains the conversion relationship between the intraoperative image and the surgical space according to the geometric feature of the target body tracer 210 in the intraoperative image and the geometric feature of the target body tracer 210 in the surgical space that is obtained by the optical tracker 120, and further converts the preoperative image into the surgical space, so that the bone fragment in the fracture can be tracked in real time and is fed back to the operator in the form of the three-dimensional model and the preoperative image for precise reduction and positioning during operation.

According to one embodiment provided by the present disclosure, the optical tracker 120 is further configured to obtain a pose of the target body tracer 210 in the actual working space in real time; and the host 110 is further configured to convert the preliminary image into the actual working space in real time, and to obtain a pose of the target body 700 in the actual working space. In this embodiment, the optical tracker 120 obtains the pose of the target body tracer 210 in real time and sends it to the host 110, and the host 110 converts the preliminary image into the actual working space in real time and obtains the pose of the target body 700 in the actual working space according to the real-time pose of the target body tracer 210. The target body 700 can be tracked in real time and is fed back to the operator in the form of the three-dimensional model and the preliminary image, so as to precisely reduce and position the target body 700 during actual work. The master control apparatus 100 tracks the target body tracer 210, and obtains the pose of the tracked target body 700 in real time. The master control apparatus may also simultaneously track a plurality of target body tracers 210 and display relative poses of a plurality of tracked target bodies 700.

When this embodiment is applied to the reduction of the bone fragment of the patient with the fracture through the medical technology, the optical tracker 120 obtains the pose of the target body tracer 210 in real time and sends it to the host 110, and the host 110 converts the preoperative image into the surgical space in real time and obtains the pose of the bone fragment in the surgical space according to the real-time pose of the target body tracer 210. The bone fragment can be tracked in real time and is fed back to the operator in the form of the three-dimensional model and the preoperative image, so as to precisely reduce and position the bone fragment in the fracture during operation. The master control trolley tracks the target body tracer 210 and obtains the pose of the tracked bone fragment in real time. The master control apparatus may also simultaneously track the plurality of target body tracers 210 and display the relative poses of the plurality of tracked bone fragments.

According to one embodiment provided by the present disclosure, the tracing apparatus 200 further includes a tool tracer 220 arranged on a tool 500; the optical tracker 120 is further configured to obtain a pose of the tool tracer 220 in the actual working space in real time; and the host 110 is further configured to obtain a pose of the tool 500 in the actual working space. In this embodiment, during navigation and reduction of the target body 700, the tool 500 for assisting in navigation is usually used, and the tool tracer 220 is correspondingly mounted on the tool 500. The optical tracker 120 obtains the pose of the tool tracer 220 in real time, and the host 110 obtains the pose of the tool 500 in the actual working space according to known relative pose of the tool tracer 220 and the tool 500. The master control apparatus 100 tracks the tool tracer 220, obtains the pose of the tool 500 where the tool tracer 220 is located in real time, and displays it, so as to guide the positioning of the tool 500.

When this embodiment is applied to the reduction of the bone fragment of the patient with the fracture through the medical technology, the tool 500 for navigation may be a cobalt drill, a positioning sleeve, a probe, etc., and the tool tracer 220 is correspondingly mounted on it. The optical tracker 120 obtains the pose of the tool tracer 220 in real time and sends it to the host 110, and the host 110 obtains the pose of the tool 500 in the surgical space according to the known relative pose of the tool tracer 220 and the tool 500 and a real-time pose of the tool tracer 220, and displays it, so as to guide the positioning of the tool 500.

According to one embodiment provided by the present disclosure, the tracing apparatus 200 further includes an operation apparatus tracer 230 arranged on the operation apparatus 600; the optical tracker 120 is further configured to obtain a pose of the operation apparatus tracer 230 in the actual working space in real time; and the host 110 is further configured to obtain a pose of the operation apparatus 600 in the actual working space, to obtain the target pose of the operation apparatus 600 in the actual working space according to a target pose of the target body 700 in the actual working space, and to control the operation apparatus 600 to move to the target pose for reduction. In this embodiment, the operation apparatus 600 is connected to the target body 700 that needs to be reduced by holding the tool 500 and a fixation pin, and the operation apparatus tracer 230 is mounted on the corresponding operation apparatus 600. The optical tracker 120 obtains the pose of the operation apparatus tracer 230 in the actual working space in real time and sends it to the host 110, and the host 110 obtains the pose of the operation apparatus 600 in the actual working space. The host 110 obtains the target pose of the target body 700 in the actual working space according to the reduction plan and the real-time pose of the target body tracer 210, and calculates the target pose of the operation apparatus 600 according to the current connection relationship between the target body 700 and the operation apparatus 600; through the communication with the operation apparatus 600, the command is sent to control the operation apparatus 600 to move to the target pose and monitor the motion state of the operation apparatus 600 in real time; and the target body 700 is operated by the operation apparatus 600 to be precisely reduced.

When this embodiment is applied to the reduction of the bone fragment of the patient with the fracture through the medical technology, the operation apparatus 600 is connected to the bone fragment that needs to be reduced by holding the tool and the fixation pin, and the operation apparatus tracer 230 is mounted on the corresponding operation apparatus 600. The optical tracker 120 obtains the pose of the operation apparatus tracer 230 in the surgical space in real time and sends it to the host 110, and the host 110 obtains the pose of the operation apparatus 600 during operation. The host 110 obtains the target pose of the bone fragment during operation according to the reduction plan and the real-time pose of the target body tracer 210, and calculates the target pose of the operation apparatus 600 according to the current connection relationship between the bone fragment and the operation apparatus 600; through the communication with the operation apparatus 600, the command is sent to control the operation apparatus 600 to move to the target pose and monitor the motion state of the operation apparatus 600 in real time; and the bone fragment is operated by the operation apparatus 600 to be precisely reduced.

According to one embodiment provided by the present disclosure, the operation apparatus 600 includes a robotic arm controller 620 and a robotic arm 610 having six or more degrees of freedom; the robotic arm 610 is connected to the robotic arm controller 620; the robotic arm controller 620 is connected to the host 110; and the operation apparatus tracer 230 is arranged on the robotic arm 610. In this embodiment, the operation apparatus 600 may include the robotic arm controller 620 and the robotic arm 610. The robotic arm controller 620 controls the robotic arm 610 to move, and the robotic arm 610 is connected to the target body that needs to be reduced by holding the tool and the fixation pin. The robotic arm 610 has six degrees of freedom, which facilitates angle and position adjustment such as overturning and horizontal movement of the target body 700. The operation apparatus tracer 230 is mounted on the corresponding robotic arm 610. The optical tracker 120 obtains the pose of the operation apparatus tracer 230 in the surgical space in real time and sends it to the host 110, and the host 110 obtains a pose of the robotic arm 610 in the actual working space. The host 110 obtains the target pose of the target body 700 during operation according to the reduction plan and the real-time pose of the target body tracer 210, and calculates a target pose of the robotic arm 610 according to a current connection relationship between the target body 700 and the robotic arm 610; through communication with the robotic arm controller 620, a command is sent to control the robotic arm 610 to move to the target pose and monitor a motion state of the robotic arm 610 in real time; and the target body 700 is operated by the robotic arm 610 to be precisely reduced.

When this embodiment is applied to the reduction of the bone fragment of the patient with the fracture through the medical technology, the robotic arm 610 is connected to the bone fragment that needs to be reduced by holding the tool and the fixation pin, and the operation apparatus tracer 230 is mounted on the corresponding robotic arm 610. The optical tracker 120 obtains the pose of the operation apparatus tracer 230 in the surgical space in real time and sends it to the host 110, and the host 110 obtains the pose of the robotic arm 610 during operation. The host 110 obtains the target pose of the bone fragment during operation according to the reduction plan and the real-time pose of the target body tracer 210, and calculates the target pose of the robotic arm 610 according to the current connection relationship between the bone fragment and the robotic arm 610; through the communication with the robotic arm controller 620, the command is sent to control the robotic arm 610 to move to the target pose and monitor the motion state of the robotic arm 610 in real time; and the bone fragment is operated by the robotic arm 610 to be precisely reduced.

In one embodiment, the master control apparatus 100 further includes a display 130 configured to display three-dimensional models for the target body 700 and the tool 500. In this embodiment, the master control apparatus 100 tracks the target body tracer 210, obtains the real-time pose of the tracked target body 700, and displays the three-dimensional model for the target body 700 in the display 130. The master control apparatus simultaneously tracks the plurality of target body tracers 210 and displays the relative poses of the plurality of tracked target bodies 700 in the display 130. The master control apparatus 100 tracks the tool tracer 220, obtains the pose of the tool 500 where the tool tracer 220 is located in real time, and displays the tool 500 in the display 130, so as to guide the positioning of the tool 500.

When this embodiment is applied to the reduction of the bone fragment of the patient with the fracture through the medical technology, the master control trolley tracks the target tracer 210, obtains the real-time pose of the tracked bone fragment, and displays the three-dimensional model for the bone fragment in the display 130. The master control trolley simultaneously tracks the plurality of target body tracers 210 and displays the relative poses of the plurality of tracked bone fragments in the display 130. The master control trolley tracks the tool tracer 220, obtains the pose of the tool 500 where the tool tracer 220 is located in real time, and displays the tool 500 in the display 130, so as to guide the positioning of the tool 500.

An embodiment of the present disclosure further provides a method for navigation and reduction operation performed using the control system for navigation and reduction operation as described in the above-mentioned embodiment, the method including:
obtaining a preliminary image and an intermediate image;
converting the preliminary image into the intermediate image according to the preliminary image and the intermediate image;
obtaining a geometric feature of a target body tracer 210 in an actual working space;
converting the preliminary image into the actual working space according to a geometric feature of the target body tracer 210 in the preliminary image and the geometric feature of the target body tracer 210 in the actual working space; and
obtaining a target pose of an operation apparatus 600 in the actual working space, and controlling the operation apparatus 600 to move to the target pose for reduction.

According to the method for navigation and reduction operation provided by this embodiment of the present disclosure, the target body tracer 210 is fixed to a target body 700 that needs to be tracked; a host 110 obtains the intermediate image and the preliminary image, and converts the preliminary image into a space of the intermediate image by matching the intermediate image with the preliminary image; an optical tracker 120 obtains the geometric feature of the target body tracer 210 in the actual working space and sends it to the host 110; and the host 110 obtains a conversion relationship between the intermediate image and the actual working space according to a geometric feature of the target body tracer 210 in the intermediate image and the geometric feature of the target body tracer 210 in the actual working space that is obtained by the optical tracker 120, and further converts the preliminary image into the actual working space. The host 110 of a master control apparatus 100 converts the preliminary image into the actual working space by matching the intermediate image with the preliminary image, and the optical tracker 120 is used to track it in real time, so that real-time three-dimensional navigation is implemented, and the target body 700 can be tracked in real time and is fed back to an operator in the form of a three-dimensional model and the preliminary image; the host 110 obtains a pose of the operation apparatus 600 in the actual working space, and calculates the target pose of the operation apparatus 600 according to a reduction plan, a real-time pose of the target body tracer 210, and a current connection relationship between the target body 700 and the operation apparatus 600; through communication with the operation apparatus 600, a command is sent to control the operation apparatus 600 to move to the target pose and monitor a motion state of the operation apparatus 600 in real time; and the target body 700 is operated by the operation apparatus 600 to be precisely reduced, so as to precisely reduce and position the target body 700 during actual work.

When this embodiment is applied to the reduction of a bone fragment of a patient with a fracture through the medical technology, the master control apparatus 100 may be a master control trolley. A preoperative imaging device 300, such as a CT machine, acquires a preoperative image of a fractured site of the patient on admission as the preliminary image and sends it to the host 110, where the preliminary image may be a three-dimensional image. An intraoperative imaging device 400, such as the CT machine, acquires an intraoperative image of the fractured site of the patient during operation as the intermediate image and sends it to the host 110, where the intermediate image may be a two-dimensional or three-dimensional image. The actual working space is a surgical space.

Firstly, the target body tracer 210 is fixed to the bone fragment that needs to be tracked, the preoperative imaging device 300 obtains the preoperative image and transmits it to the host 110, the intraoperative imaging device 400 obtains the intraoperative image and transmits it to the host 110, and the host 110 converts the preoperative image into the space of the intraoperative image by matching the preoperative image with the intraoperative image through image or point cloud registration. The host 110 obtains the conversion relationship between the intraoperative image and the surgical space according to the geometric feature of the target body tracer 210 in the intraoperative image and the geometric feature of the target body tracer 210 in the surgical space that is obtained by the optical tracker 120, and further converts the preoperative image into the surgical space, so that the bone fragment in the fracture can be tracked in real time and is fed back to the operator in the form of the three-dimensional model and the preoperative image for precise reduction and positioning during operation.

According to an embodiment provided by the present disclosure, the method for navigation and reduction operation in this embodiment of the present disclosure further includes:
obtaining a real-time pose of the target body tracer 210; and
obtaining a three-dimensional model for a target body 700 on the basis of converting the preliminary image into the actual working space.

In this embodiment, the optical tracker 120 obtains the pose of the target body tracer 210 in real time and sends it to the host 110, and the host 110 converts the preliminary image into the actual working space in real time and obtains the pose of the target body 700 in the actual working space according to the real-time pose of the target body tracer 210. The target body 700 can be tracked in real time and is fed back to the operator in the form of the three-dimensional model and the preliminary image, so as to precisely reduce and position the target body 700 during actual work. The master control apparatus 100 tracks the target body tracer 210 and obtains the pose of the tracked target body 700 in real time. The master control apparatus may also simultaneously track a plurality of target body tracers 210 and display relative poses of a plurality of tracked target bodies 700. A tracking method is to repeat the process in the above-mentioned embodiment.

The master control apparatus 100 tracks the target body tracer 210, obtains a real-time pose of the tracked target body 700, and displays the three-dimensional model for the target body 700 in a display 130. The master control apparatus simultaneously tracks the plurality of target body tracers 210 and displays the relative poses of the plurality of tracked target bodies 700 in the display 130.

When this embodiment is applied to the reduction of the bone fragment of the patient with the fracture through the medical technology, the optical tracker 120 obtains the pose of the target body tracer 210 in real time and sends it to the host 110, and the host 110 converts the preoperative image into the surgical space in real time and obtains the pose of the bone fragment in the surgical space according to the real-time pose of the target body tracer 210. The bone fragment can be tracked in real time and is fed back to the operator in the form of the three-dimensional model and the preoperative image, so as to precisely reduce and position the bone fragment in the fracture during operation. The master control trolley tracks the target body tracer 210 and obtains the pose of the tracked bone fragment in real time. The master control apparatus may also simultaneously track the plurality of target body tracers 210 and display the relative poses of the plurality of tracked bone fragments. The master control trolley tracks the target body tracer 210, obtains the real-time pose of the tracked bone fragment, and displays the three-dimensional model for the bone fragment in the display 130. The master control trolley simultaneously tracks the plurality of target body tracers 210 and displays the relative poses of the plurality of tracked bone fragments in the display 130.

According to an embodiment provided by the present disclosure, the method for navigation and reduction operation in this embodiment of the present disclosure further includes:
obtaining a real-time pose of a tool tracer 220; and
obtaining a pose of the tool 500 in the actual working space and a three-dimensional model for the tool 500 according to a relative pose of the tool tracer 220 and the tool 500 and the real-time pose of the tool tracer 220.

In this embodiment, during navigation and reduction of the target body 700, the tool 500 for assisting in navigation is usually used, and the tool tracer 220 is correspondingly mounted on the tool 500. The optical tracker 120 obtains the pose of the tool tracer 220 in real time, and the host 110 obtains the pose of the tool 500 in the actual working space according to known relative pose of the tool tracer 220 and the tool 500. The master control apparatus 100 tracks the tool tracer 220, obtains the pose of the tool 500 where the tool tracer 220 is located in real time, and displays it, so as to guide the positioning of the tool 500. The master control apparatus 100 tracks the tool tracer 220, obtains the pose of the tool 500 where the tool tracer 220 is located in real time, and displays the tool 500 in the display 130, so as to guide the positioning of the tool 500.

When this embodiment is applied to the reduction of the bone fragment of the patient with the fracture through the medical technology, the tool 500 for navigation may be a cobalt drill, a positioning sleeve, a probe, etc., and the tool tracer 220 is correspondingly mounted on it. The optical tracker 120 obtains the pose of the tool tracer 220 in real time and sends it to the host 110, and the host 110 obtains the pose of the tool 500 in the surgical space according to the known relative pose of the tool tracer 220 and the tool 500 and a real-time pose of the tool tracer 220, and displays it, so as to guide the positioning of the tool 500. The master control trolley tracks the tool tracer 220, obtains the pose of the tool 500 where the tool tracer 220 is located in real time, and displays the tool 500 in the display 130, so as to guide the positioning of the tool 500.

According to an embodiment provided by the present disclosure, the method for navigation and reduction operation in this embodiment of the present disclosure further includes:
obtaining a real-time pose of an operation apparatus tracer 230;
obtaining a pose of the operation apparatus 600 in the actual working space according to relative pose of the operation apparatus tracer 230 and the operation apparatus 600 and the real-time pose of the operation apparatus tracer 230;
obtaining a target pose of the target body 700 in the actual working space;
obtaining the target pose of the operation apparatus 600 according to a connection relationship between the target body 700 and the operation apparatus 600; and
controlling the operation apparatus 600 to move to the target pose.

In this embodiment, the operation apparatus 600 is connected to the target body 700 that needs to be reduced by holding the tool 500 and a fixation pin, and the operation apparatus tracer 230 is mounted on the corresponding operation apparatus 600. The optical tracker 120 obtains the pose of the operation apparatus tracer 230 in the actual working space in real time and sends it to the host 110, and the host 110 obtains the pose of the operation apparatus 600 in the actual working space. The host 110 obtains the target pose of the target body 700 in the actual working space according to the reduction plan and the real-time pose of the target body tracer 210, and calculates the target pose of the operation apparatus 600 according to the current connection relationship between the target body 700 and the operation apparatus 600; through the communication with the operation apparatus 600, the command is sent to control the operation apparatus 600 to move to the target pose and monitor the motion state of the operation apparatus 600 in real time; and the target body 700 is operated by the operation apparatus 600 to be precisely reduced.

When this embodiment is applied to the reduction of the bone fragment of the patient with the fracture through the medical technology, the operation apparatus 600 may include a robotic arm controller 620 and a robotic arm 610. The robotic arm controller 620 controls the robotic arm 610 to move, and the robotic arm 610 is connected to the bone fragment that needs to be reduced by holding the tool 500 and the fixation pin. The operation apparatus tracer 230 is mounted on the corresponding robotic arm 610. The optical tracker 120 obtains the pose of the operation apparatus tracer 230 in the surgical space in real time and sends it to the host 110, and the host 110 obtains the pose of the robotic arm 610 during operation. The host 110 obtains the target pose of the bone fragment during operation according to the reduction plan and the real-time pose of the target body tracer 210, and calculates a target pose of the robotic arm 610 according to a current connection relationship between the bone fragment and the robotic arm 610; through communication with the robotic arm controller 620, a command is sent to control the robotic arm 610 to move to the target pose and monitor a motion state of the robotic arm 610 in real time; and the bone fragment is operated by the robotic arm 610 to be precisely reduced.

Finally, it should be noted that the above embodiments are merely used to illustrate the technical solutions of the present disclosure, but not to limit them; although the present disclosure has been described in detail with reference to the above-mentioned embodiments, those of ordinary skill in the art should understand that: they may still make modifications to the technical solutions described in the above-mentioned embodiments, or make equivalent substitutions to some of the technical features; and these modifications or substitutions do not make the essence of the corresponding technical solutions depart from the spirit and scope of the technical solutions of the embodiments of the present disclosure.

1. A control system for navigation and reduction operation, comprising a master control apparatus, a tracing apparatus, and an operation apparatus, wherein the master control apparatus comprises a host and an optical tracker; the tracing apparatus comprises a target body tracer arranged on a target body; the optical tracker is configured to obtain a geometric feature of the target body tracer in an actual working space; the host is configured to convert a preliminary image into an intermediate image by matching the preliminary image with the intermediate image, and to convert the preliminary image into the actual working space according to a geometric feature of the target body tracer in the intermediate image and the geometric feature of the target body tracer in the actual working space; and the host is further configured to obtain a target pose of the operation apparatus in the actual working space, and to control the operation apparatus to move to the target pose for reduction.

2. The control system for navigation and reduction operation according to claim 1, wherein the optical tracker is further configured to obtain a pose of the target body tracer in the actual working space in real time; and the host is further configured to convert the preliminary image into the actual working space in real time, and to obtain a pose of the target body in the actual working space.

3. The control system for navigation and reduction operation according to claim 2, wherein the tracing apparatus further comprises a tool tracer arranged on a tool; the optical tracker is further configured to obtain a pose of the tool tracer in the actual working space in real time; and the host is further configured to obtain a pose of the tool in the actual working space.

4. The control system for navigation and reduction operation according to claim 3, wherein the tracing apparatus further comprises an operation apparatus tracer arranged on the operation apparatus; the optical tracker is further configured to obtain a pose of the operation apparatus tracer in the actual working space in real time; and the host is further configured to obtain a pose of the operation apparatus in the actual working space, to obtain the target pose of the operation apparatus in the actual working space according to a target pose of the target body in the actual working space, and to control the operation apparatus to move to the target pose for reduction.

5. The control system for navigation and reduction operation according to claim 4, wherein the operation apparatus comprises a robotic arm controller and a robotic arm having six or more degrees of freedom; the robotic arm is connected to the robotic arm controller; the robotic arm controller is connected to the host; and the operation apparatus tracer is arranged on the robotic arm.

6. A method for navigation and reduction operation performed using the control system for navigation and reduction operation according to claim 1, the method comprising:
obtaining a preliminary image and an intermediate image;
converting the preliminary image into the intermediate image according to the preliminary image and the intermediate image;
obtaining a geometric feature of a target body tracer in an actual working space;
converting the preliminary image into the actual working space according to a geometric feature of the target body tracer in the preliminary image and the geometric feature of the target body tracer in the actual working space; and
obtaining a target pose of an operation apparatus in the actual working space, and controlling the operation apparatus to move to the target pose for reduction.

7. The method for navigation and reduction operation according to claim 6, further comprising:
obtaining a real-time pose of the target body tracer; and
obtaining a three-dimensional model for a target body on the basis of converting the preliminary image into the actual working space.

8. The method for navigation and reduction operation according to claim 7, further comprising:
obtaining a real-time pose of a tool tracer; and
obtaining a pose of the tool in the actual working space and a three-dimensional model for the tool according to a relative pose of the tool tracer and the tool and the real-time pose of the tool tracer.

9. The method for navigation and reduction operation according to claim 8, further comprising:
obtaining a real-time pose of an operation apparatus tracer; and
obtaining a pose of the operation apparatus in the actual working space according to a relative pose of the operation apparatus tracer and the operation apparatus and the real-time pose of the operation apparatus tracer.

10. The method for navigation and reduction operation according to claim 9, further comprising:
obtaining a target pose of the target body in the actual working space;
obtaining the target pose of the operation apparatus according to a connection relationship between the target body and the operation apparatus; and
controlling the operation apparatus to move to the target pose.
